(19) United States
(12) Patent Application Publication
(10) Pub. No.: US 2008/0097325 A1
(43) Pub. Date: Apr. 24, 2008

(54) Method and apparatus for controlling the injection rate of a drug solution in a cartridge type motor-driven dental injection syringe

(75) Inventors: Fumio Tanaka, Tokyo (JP); Yoshihiko Kawasaki, Tokyo (JP); Mitsuhiro Haraguchi, Tokyo (JP); Ikuo Kitagawa, Matsudo-shi (JP); Yoshinori Kato, Matsudo-shi (JP); Renji Hayashi, Matsudo-shi (JP)

Correspondence Address: CHAPMAN AND CUTLER, 111 WEST MONROE STREET, CHICAGO, IL, 60603, US

(73) Assignee: Showa Yakuhin Kako Co., Ltd., Chuo-ku (JP)

(21) Appl. No.: 11/895,496

(22) Filed: Aug. 24, 2007

Related U.S. Application Data
(63) Continuation of application No. PCT/JP05/19989, filed on Oct. 31, 2005.

(30) Foreign Application Priority Data
Feb. 24, 2005 (JP) 2005-049233

(51) Int. Cl. A61M 5/20

(52) U.S. Cl. 604/154

(57) ABSTRACT

Disclosed are a method and apparatus for controlling the injection rate of an anesthetic solution in a cartridge type motor-driven injection syringe for dental use, particularly beginning with the “Low” rate injection at the outset, and switching to the “Medium” or “High” injection rate at an arbitrarily selected time in the course of injection.

The speed of revolution of the electric motor is changed among the low, medium and high speed, and a two-step operable type of switch is used as a starter switch 13. The first depression of the starter switch makes the electric motor 9 start at the low speed, thereby making the plunger rubber 5 initially move at the “Low Speed” in the cartridge 6 via the plunger rod 12, and the second depression of the switch makes the electric motor 9 run at the low, medium or high speed, and accordingly the plunger rod moves at the selected “Low Speed”, “Medium Speed” or “High Speed”.

### BACKGROUND OF THE INVENTION

1. Field of the Invention

The present invention relates to a cartridge type motor-driven injection syringe for dental use, and particularly to a method and apparatus for controlling the injection rate of a drug solution such as an anesthetic solution in a motor-driven injection syringe.

2. Prior Art

The local anesthesia is indispensable for removal of gingiva, preparation of cavities, extraction of tooth and the like. Nerves to be anaesthetized run in the jaw bone or tooth, and therefore, usually the anesthetic solution is not applied directly to the nerves. In almost all cases the injection needle is inserted in the soft tissue, periodontal ligament, or near the bone close to a selected nerve, and then the anesthetic solution is locally injected, allowing the anesthetic solution to spread all over the local area, thereby making the anesthetic solution work on the nerve, which is called “Infiltration Anesthesia”.

Anesthetic-filled cartridges of different sizes (1.0 ml or 1.8 ml) are commercially available. In use such a cartridge is attached to a manually operable injection syringe, and a double-pointed injection needle is fixed to the injection syringe with one pointed end penetrated a rubber disc, by which the end of the cartridge is hermetically sealed. The cartridge has a plunger rubber movably fitted in the rear end of the cartridge for pushing the anesthetic solution out of the cartridge through the injection needle. A plunger rod of the injection syringe is operated with fingers to push the plunger rubber, thus applying a pressure to the anesthetic solution for discharging out of the cartridge. As may be realized, such intraligamentary injection requires application of a relatively high pressure to the injection syringe. In view of this a variety of motor-driven injection syringes for dental use have been developed as a substitute for the manually operable type of injection syringe, and have been commercially available.

The motor-driven injection syringe is basically equipped with a final reduction gear to reduce the speed of revolution of the electric motor, and a rack and pinion to convert the so reduced revolution into the linear movement of the plunger rod. To meet the demand for controlling the injection rate of the anesthetic solution in terms of the linear speed of the plunger rod (the injection rate being defined as a length of time involved for injecting the anesthetic solution of 1 ml) the present applicant has developed and marketed motor-driven injection syringes, which are so designed that dentists may choose among three different injection rates, that is, Low (200 secs/1 ml), Medium (100 secs/1 ml) and High (60 secs/1 ml) injection rates. The speed of revolution of the electric motor, therefore, is changed three stepwise, so that the plunger rod may travel at any one speed selected among Low (9.5 mm/min.), Medium (14.5 mm/min.) and High (23.0 mm/min.) speeds.

In dental local anesthesia patients feel pain twice, that is, at the time of needle insertion into the oral tissue and at the time of the flow of the anesthetic solution into the oral tissue. Nowadays injection needles have been improved such that the pain which patients feel in injection significantly lessens. Specifically, injection needles are reduced in outer diameter and thickness, and increased in inner diameter. Incidentally it is realized that the pain which patients feel at the beginning of injection lessens if the anesthetic solution is injected at a reduced flow rate. In view of this the motor-driven injection syringe is set at such a decreased speed that the pain at the beginning of the injection may noticeably lessen. The length of time involved for the injection can be shortened by setting the injection rate at the “Medium” or “High” although the problem of initial pain remains unsolved.

With a view to reaching a compromise some motor-driven injection syringes are equipped with automatic control means, which is programmed as for example follows: the injection rate gradually rises from zero for a predetermined length of time (3 seconds for low speed; 7 seconds for medium speed; and 10 seconds for high speed), and then, the injection rate is automatically changed for the Low rate (for example, 180 seconds/1 ml), the Medium rate (for example, 97 seconds/1 ml) or the High rate (for example, 70 seconds/1 ml) set by the dentist, and is kept at the set value for the remaining length of time. This effectively prevents patients against the initial pain. The programming of injecting operation, however, does not allow dentists to arbitrarily adjust the initial injection period prior to the switching to the Low, Medium or High rate.

### SUMMARY OF THE INVENTION

One object of the present invention is to provide a method and apparatus for controlling the injection rate of an anaesthetic solution in a cartridge type motor-driven injection syringe for dental use wherein the initial injection begins with the “Low” injection rate, and the consequent injection is made by switching from “Low” injection rate to the “Medium” or “High” injection rate at an arbitrarily selected time.

Another object of the present invention is to provide a method and apparatus for controlling the injection rate of an anaesthetic solution in a cartridge type motor-driven injection syringe for dental use wherein that the “low” injection rate for the initial injection can also be maintained for the whole period of injection.

To attain these objects, the present invention provides a method for controlling the injection rate of an anaesthetic solution in a cartridge type motor-driven injection syringe for dental use, the motor-driven injection syringe being so designed that the electric motor is operatively connected through a reduction gear to a rack and pinion to slowly move a plunger rod and hence a plunger rubber in the cartridge, thereby ejecting the anaesthetic solution through an injection needle, characterized in that the electric motor is adapted to be switched stepwise in speed of revolution between the Low, Medium and High Speed, the starter switch is a two-step operable switch including the first depression for starting the electric motor at the Low Speed and hence, moving the plunger rod so as to push the plunger rubber in the cartridge at the initial “Low Speed”, and the second depression for making the electric motor run at the “Low Speed”, “Medium Speed” or “High Speed” as selected, to move the plunger rod at the “Low Speed”, “Medium Speed” or “High Speed”.

In another aspect, the present invention provides an apparatus for controlling the injection rate of an anaesthetic solution in a cartridge type motor-driven injection syringe for dental use, the motor-driven injection syringe comprising a housing with a nose, a cartridge holder to accommodate an anaesthetic solution-filled cartridge having a rubber disc and a plunger rubber fitted therein, the cartridge holder being detachably attached to the nose; a double-pointed injection needle detachably attached to the end of the cartridge holder, penetrating the rubber disc; a plunger rod adapted to be linearly displaced by the rack and pinion operatively connected through a final reduction gear to the electric motor, thereby ejecting the anaesthetic solution from the cartridge through the injection needle, and a starter switch for starting the electric motor, characterized in that the starter switch comprises two switches responsive to first and second depressions for sequential operation, the motor-driven injection syringe further comprises a speed selector switch for making a desired selection among the “Low Speed”, “Medium Speed” and “High Speed” to set the electric motor for the desired running speed, a speed changing circuit and a speed control circuit responsive to the first depression of the starter switch to turn on for making the electric motor run at the low speed, and responsive to the second depression to turn on for making the electric motor run at the “Low Speed”, “Medium Speed” or “High Speed” dependent on which circuit the speed selector switch is connected to, the “Low Speed Circuit”, “Medium Speed Circuit” or “High Speed Circuit”.

Other objects and advantages of the present invention will be understood from the following description of one preferred embodiment of the present invention as shown in the drawings.

### BRIEF DESCRIPTION OF THE DRAWINGS

FIG. 1 is a perspective view of a cartridge type of motor-driven injection syringe for dental use according to one embodiment of the present invention;

FIG. 2 is a longitudinal section of the cartridge type of motor-driven injection syringe for dental use:

FIG. 3 is a plane view of the motor-driven injection syringe of FIG. 2, partially broken to show inside; and

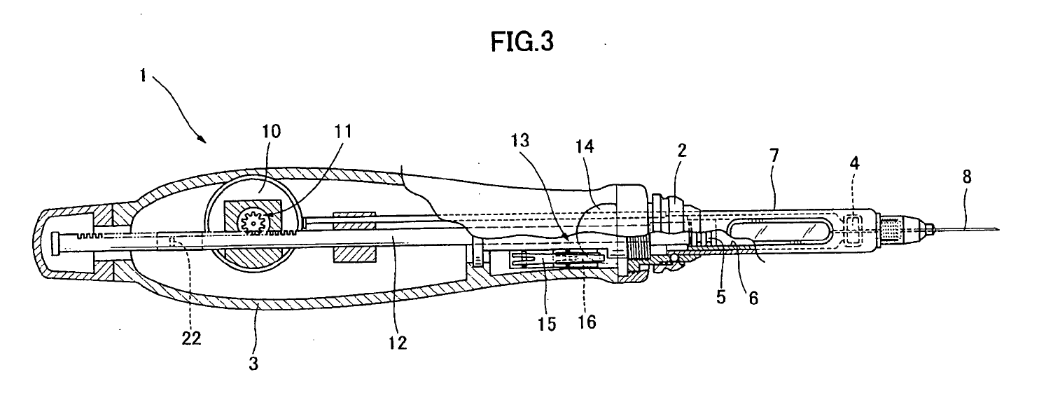

FIG. 4 is a block diagram showing the circuits for controlling the anaesthetic injection rate in the motor-driven injection syringe.

### DESCRIPTION OF THE PREFERRED EMBODIMENT

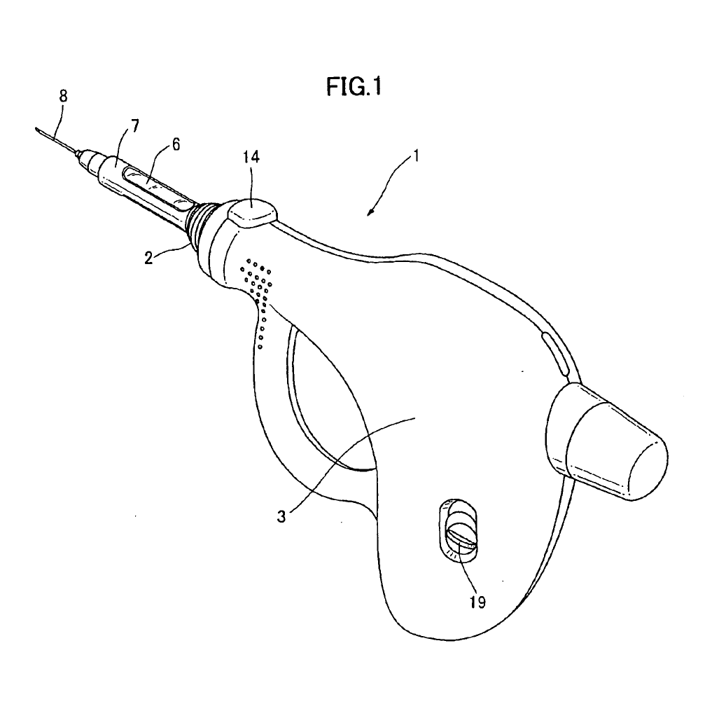

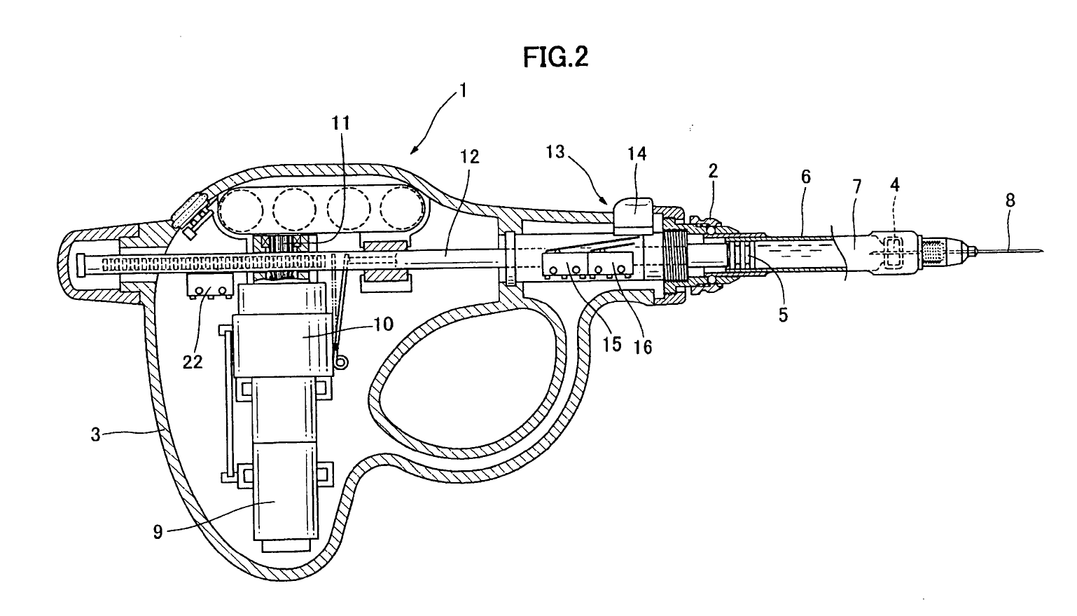

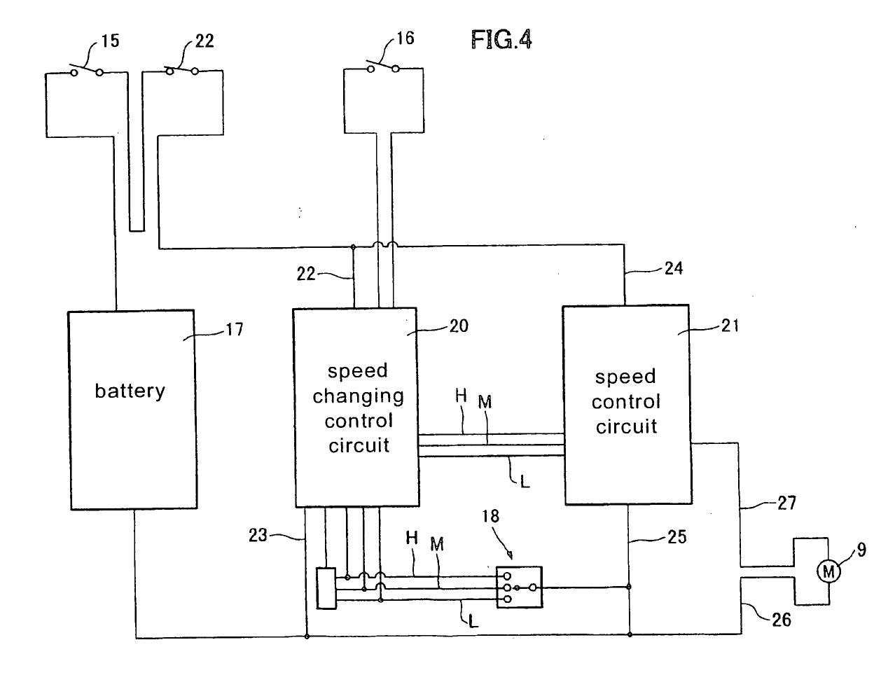

FIG. 1 shows a cartridge type motor-driven injection syringe 1 for dental use according to one embodiment of the present invention. As can be seen from FIGS. 1 and 2, the motor-driven injection syringe 1 comprises a housing 3 with a nose 2, a cartridge holder 7 to accommodate an anaesthetic solution-filled cartridge 6, which has a rubber disc 4 and a plunger rubber 5 fitted therein, a double-pointed injection needle 8 detachably attached to the end of the cartridge holder 7, penetrating the rubber disc 4; a plunger rod 12 adapted to be linearly displaced by a rack and pinion 11 operatively connected through a final reduction gear 10 to an electric motor 9, thereby ejecting the anaesthetic solution from the cartridge 6 through the injection needle 8, a starter switch 13 for starting the electric motor 9, and a button 14 for operating the starter switch 13. The starter switch 13 comprises two sequential switches 15 and 16 responsive to first and second depressions for sequential operation, as described below with reference to a block diagram of FIG. 4.

In the block diagram, an electric power supply or battery is indicated by 17. There is provided a speed selector switch 18 permiting a dentist to make a selection among the “Low Speed Circuit”, “Medium Speed Circuit” and “High Speed Circuit” for rotating the electric motor 9 accordingly. The speed selector switch 18 has a knob slide 19 accessible from the outside of the cartridge type motor-driven injection syringe. A speed changing circuit 20 and a speed control circuit 21 are connected to each other by wires L, M and H in a central process unit. When the plunger rod 12 travels the forward stroke, a limit switch 22 opens the circuit to make the electric motor 9 stop.

When the first depression makes the switch 15 turn on, the speed changing circuit 20 is supplied with electricity from the battery 17 via the wires 22 and 23, and likewise the speed control circuit 21 is supplied with electricity from the battery 17 via the leads 24 and 25. The control circuit 20 sends a “Low Speed” signal to the speed control circuit 21 via the wire L, thereby making the electric motor 9 which is connected to the power supply 17 run at the “Low Speed”. The speed selector switch 18 makes a selection among the “Low Speed Circuit”, “Medium Speed Circuit” and “High Speed Circuit” by moving the knob slide 19. Subsequently, the second depression makes the switch 16 turn on. Then, the speed changing circuit 20 sends the “Low Speed”, “Medium Speed” or “High Speed” signal to the speed control circuit 21 to make the electric motor 9 run at the selected speed of revolution. In any event, the speed of revolution of the electric motor 9 is reduced by the final reduction gear 10, and the plunger rod 12 is linearly moved by the rack and pinion at the reduced speed to push the plunger rubber 5, thereby ejecting the anaesthetic solution from the cartridge 6 through the injection needle 8.

In use, the knob slide 19 is moved such that the speed selector switch 18 select the “Medium Speed” or the “High Speed”. Then, the first depression of the switch 13 causes the injection of the anaesthetic solution at the reduced injection rate. When it is confirmed that the patient is anethetized in an injection area, the dentist gives the second push to the switch 13 to inject the anaesthetic solution at the medium or high injection rate. In consideration of the patient's condition the dentist can adjust the length of time involved for the low injection rate. Thus, the dentist can shorten or lengthen the length of time involved for the anaesthetic injection as desired.

List of Reference Numbers:

1 cartridge type motor-driven dental injection syringe.
2 nose
3 housing
4 rubber disc
5 plunger rubber
6 cartridge
7 cartridge holder
8 double-pointed injection needle
9 electric motor
10 final reduction gear
11 rack and pinion
12 plunger rod
13 two-push operable type of switch
14 button
15 switch
16 switch
17 battery
18 speed selector switch
19 knob slide
20 speed changing control circuit
21 speed control circuit
22 limit switch

What is claimed is:

1. A method for controlling the injection rate of an anaesthetic solution in a cartridge type motor-driven injection syringe for dental use, the motor-driven injection syringe being so designed that the electric motor is operatively connected through a reduction gear to a rack and pinion to slowly move a plunger rod and hence a plunger rubber in the cartridge, thereby ejecting the anaesthetic solution through an injection needle, characterized in that the electric motor is adapted to be switched stepwise in speed of revolution between the Low, Medium and High Speed, the starter switch is a two-step operable switch including to the first depression for starting the electric motor at the Low Speed and hence, moving the plunger rod so as to push the plunger rubber in the cartridge at the initial “Low Speed”, and the second depression for making the electric motor run at the “Low Speed”, “Medium Speed” or “High Speed” as selected, to move the plunger rod at the “Low Speed”, “Medium Speed” or “High Speed”.

2. An apparatus for controlling the injection rate of an anaesthetic solution in a cartridge type motor-driven injection syringe for dental use, the motor-driven injection syringe comprising a housing with a nose, a cartridge holder to accommodate an anaesthetic solution-filled cartridge having a rubber disc and a plunger rubber fitted therein, the cartridge holder being detachably attached to the nose; a double-pointed injection needle detachably attached to the end of the cartridge holder, penetrating the rubber disc; a plunger rod adapted to be linearly displaced by the rack and pinion operatively connected through a final reduction gear to the electric motor, thereby ejecting the anaesthetic solution from the cartridge through the injection needle, and a starter switch for starting the electric motor, characterized in that the starter switch comprises two switches responsive to first and second depressions for sequential operation, the motor-driven injection syringe further comprises:
a speed selector switch for making a desired selection among the “Low Speed”, “Medium Speed” and “High Speed” to set the electric motor for the desired running speed,
a speed changing circuit and a speed control circuit responsive to the first depression of the starter switch to turn on for making the electric motor run at the low speed, and responsive to the second depression to turn on for making the electric motor run at the “Low Speed”, “Medium Speed” or “High Speed” dependent on which circuit the speed selector switch is connected to, the “Low Speed Circuit”, “Medium Speed Circuit” or “High Speed Circuit”.
